(19) United States
(12) Patent Application Publication
(10) Pub. No.: US 2010/0158866 A1
(43) Pub. Date: Jun. 24, 2010

(54) PREDICTION OF HCV TREATMENT RESPONSE

(76) Inventor: Yonghong Zhu, Mountain View, CA (US)

Correspondence Address: Grant D. Green;Patent Law Department,, M/S A2-250, Roche Palo Alto LLC, 3431 Hillview Avenue, Palo Alto, CA, 94304, US

(21) Appl. No.: 12/634,968

(22) Filed: Dec. 10, 2009

Related U.S. Application Data
(60) Provisional application No. 61/138,585, filed on Dec. 18, 2008.

(51) Int. Cl. A61K 38/21

(52) U.S. Cl. 424/85.7; 424/85.4

(57) ABSTRACT

The present invention is based on the discovery that in patients infected with Hepatitis C Virus Genotype 1 (HCV-1) or Genotype 4 (HCV-4), a beneficial response to a treatment that includes interferon alpha, ribavirin and a HCV polymerase inhibitor could be predicted if the patient's HCV RNA level becomes undetectable in as short as two weeks post treatment.

### CROSS REFERENCE TO RELATED INVENTIONS

This application claims the benefit of priority of U.S. Provisional Patent Application Ser. No. 61/138,585, filed Dec. 18, 2008, which is incorporated herein by reference in its entirety.

### FIELD OF THE INVENTION

The present invention relates to methods that useful for predicting the response of hepatitis C virus infected patients to pharmacological treatment.

### BACKGROUND OF THE INVENTION

Hepatitis C virus (HCV) is a major health problem and the leading cause of chronic liver disease throughout the world. (Boyer, N. et al. J. Hepatol. 2000 32:98-112). Patients infected with HCV are at risk of developing cirrhosis of the liver and subsequent hepatocellular carcinoma and, hence, HCV is the major indication for liver transplantation.

According to the World Health Organization, there are more than 200 million infected individuals worldwide, with at least 3 to 4 million people being infected each year. Once infected, about 20% of people clear the virus, but the rest can harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring. Current treatments for HCV infection, which are restricted to immunotherapy with recombinant interferon-α alone or in combination with the nucleoside analog ribavirin, are of limited clinical benefit as resistance develops rapidly. There is an urgent need for improved therapeutic agents that effectively combat chronic HCV infection

HCV has been classified as a member of the virus family Flaviviridae that includes the genera flaviviruses, pestiviruses, and hepaciviruses which includes hepatitis C viruses (Rice, C. M., Flaviviridae: The viruses and their replication, in: Fields Virology, Editors: Fields, B. N., Knipe, D. M., and Howley, P. M., Lippincott-Raven Publishers, Philadelphia, Pa., Chapter 30, 931-959, 1996). HCV is an enveloped virus containing a positive-sense single-stranded RNA genome of approximately 9.4 kb. The viral genome consists of a 5′-untranslated region (UTR), a long open reading frame (ORF) encoding a polyprotein precursor of approximately 3011 amino acids, and a short 3′ UTR. The 5′ UTR is the most highly conserved part of the HCV genome and is important for the initiation and control of polyprotein translation.

Genetic analysis of HCV has identified six main genotypes showing a >30% divergence in their DNA sequence. Each genotype contains a series of more closely related subtypes which show a 20-25% divergence in nucleotide sequences (Simmonds, P. 2004 J. Gen. Virol. 85:3173-88). More than 30 subtypes have been distinguished. In the US approximately 70% of infected individuals have Type 1a and 1b infection. Type 1b is the most prevalent subtype in Asia. (X. Forms and J. Bukh, Clinics in Liver Disease 1999 3:693-716; J. Bukh et al., Semin. Liv. Dis. 1995 15:41-63). Unfortunately Type 1 infections are more resistant to therapy than either the type 2 or 3 genotypes (N. N. Zein, Clin. Microbiol. Rev., 2000 13:223-235).

The genetic organization and polyprotein processing of the nonstructural protein portion of the ORF of pestiviruses and hepaciviruses is very similar. These positive stranded RNA viruses possess a single large ORF encoding all the viral proteins necessary for virus replication. These proteins are expressed as a polyprotein that is co- and post-translationally processed by both cellular and virus-encoded proteinases to yield the mature viral proteins. The viral proteins responsible for the replication of the viral genome RNA are located within approximately the carboxy-terminal. Two-thirds of the ORF are termed nonstructural (NS) proteins. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B.

The NS proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al. Nature 1988 333:22; Bazan and Fletterick Virology 1989 171:637-639; Gorbalenya et al. Nucleic Acid Res. 1989 17.3889-3897). Similarly, the NS5B proteins of pestiviruses and hepaciviruses have the motifs characteristic of RNA-directed RNA polymerases (Koonin, E. V. and Dolja, V. V. Crit. Rev. Biochem. Molec. Biol. 1993 28:375-430).

The actual roles and functions of the NS proteins of pestiviruses and hepaciviruses in the lifecycle of the viruses are directly analogous. In both cases, the NS3 serine proteinase is responsible for all proteolytic processing of polyprotein precursors downstream of its position in the ORF (Wiskerchen and Collett Virology 1991 184:341-350; Bartenschlager et al. J. Virol. 1993 67:3835-3844; Eckart et al. Biochem. Biophys. Res. Comm. 1993 192:399-406; Grakoui et al. J. Virol. 1993 67:2832-2843; Grakoui et al. Proc. Natl. Acad. Sci. USA 1993 90:10583-10587; Ilijikata et al. J. Virol. 1993 67:4665-4675; Tome et al. J. Virol. 1993 67:4017-4026). The NS4A protein, in both cases, acts as a cofactor with the NS3 serine protease (Bartenschlager et al. J. Virol. 1994 68:5045-5055; Failla et al. J. Virol. 1994 68: 3753-3760; Xu et al. J. Virol. 1997 71:53 12-5322). The NS3 protein of both viruses also functions as a helicase (Kim et al. Biochem. Biophys. Res. Comm. 1995 215: 160-166; Jin and Peterson Arch. Biochem. Biophys. 1995, 323:47-53; Warrener and Collett J. Virol. 1995 69:1720-1726). Finally, the NS5B proteins of pestiviruses and hepaciviruses have the predicted RNA-directed RNA polymerases activity (Behrens et al. EMBO 1996 15:12-22; Lechmann et al. J. Virol. 1997 71:8416-8428; Yuan et al. Biochem. Biophys. Res. Comm. 1997 232:231-235; Hagedorn, PCT WO 97/12033; Zhong et al. J. Virol. 1998 72:9365-9369).

Currently there are a limited number of approved therapies are currently available for the treatment of HCV infection. New and existing therapeutic approaches to treating HCV and inhibition of HCV NS5B polymerase have been reviewed: R. G. Gish, Sem. Liver. Dis., 1999 19:5; Di Besceglie, A. M. and Bacon, B. R., Scientific American, October: 1999 80-85; G. Lake-Bakaar, Current and Future Therapy for Chronic Hepatitis C Virus Liver Disease, Curr. Drug Targ. Infect Dis. 2003 3(3):247-253; P. Hoffmann et al., Recent patents on experimental therapy for hepatitis C virus infection (1999-2002), Exp. Opin. Ther. Patents 2003 13(11):1707-1723; F. F. Poordad et al. Developments in Hepatitis C therapy during 2000-2002, Exp. Opin. Emerging Drugs 2003 8(1):9-25; M. P. Walker et al., Promising Candidates for the treatment of chronic hepatitis C, Exp. Opin. Investig. Drugs 2003 12(8):1269-1280; S.-L. Tan et al., Hepatitis C Therapeutics: Current Status and Emerging Strategies, Nature Rev. Drug Discov. 2002 1:867-881; R. De Francesco et al. Approaching a new era for hepatitis C virus therapy: inhibitors of the NS3-4A serine protease and the NS5B RNA-dependent RNA polymerase, Antiviral Res. 2003 58:1-16; Q. M. Wang et al. Hepatitis C virus encoded proteins: targets for antiviral therapy, Drugs of the Future 2000 25(9):933-8-944; J. A. Wu and Z. Hong, Targeting NS5B-Dependent RNA Polymerase for Anti-HCV Chemotherapy Cur. Drug Targ.-Inf Dis. 2003 3:207-219. The reviews cite compounds presently in various stages of the development process are hereby incorporated by reference in their entirety.

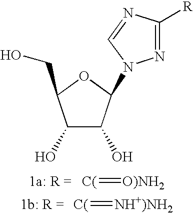

Ribavirin (1a; 1-((2R,3R,4S,5R)-3,4-Dihydroxy-5-hydroxymethyl-tetrahydro-furan-2-yl)-1H-[1,2,4]triazole-3-carboxylic acid amide; Virazole®) is a synthetic, non-interferon-inducing, broad spectrum antiviral nucleoside analog. Ribavirin has in vitro activity against several DNA and RNA viruses including Flaviviridae (Gary L. Davis, Gastroenterology 2000 118:S104-S114). In monotherapy ribavirin reduces serum amino transferase levels to normal in 40% of patients, but it does not lower serum levels of HCV-RNA. Ribavirin also exhibits significant toxicity and is known to induce anemia. Ribavirin is an inhibitor of inosine monophosphate dehydrogenase. Ribavirin is not approved in monotherapy against HCV but the compound is approved in combination therapy with interferon α-2a and interferon α-2b. Viramidine 1b is a prodrug converted to 1a in hepatocytes.

Interferons (IFNs) have been available for the treatment of chronic hepatitis for nearly a decade. IFNs are glycoproteins produced by immune cells in response to viral infection. Two distinct types of interferon are recognized: Type 1 includes several interferon alphas and one interferon β, type 2 includes interferon γ. Type 1 interferon is produced mainly by infected cells and protects neighboring cells from de novo infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary. Cessation of therapy results in a 70% relapse rate and only 10-15% exhibit a sustained virological response with normal serum alanine transferase levels. (L.-B. Davis, supra)

One limitation of early IFN therapy was rapid clearance of the protein from the blood. Chemical derivatization of IFN with polyethyleneglycol (PEG) has resulted in proteins with substantially improved pharmacokinetic properties. Pegasys® is a conjugate interferon α-2a and a 40 kD branched mono-methoxy PEG and Peg-Intron® is a conjugate of interferon α-2b and a 12 kD mono-methoxy PEG. (B. A. Luxon et al., Clin. Therap. 2002 24(9):13631383; A. Kozlowski and J. M. Harris, J. Control. Release, 2001 72:217-224).

Interferon α-2a and interferon α-2b are currently approved as monotherapy for the treatment of HCV. Roferon-A® (Roche) is the recombinant form of interferon α-2a. Pegasys® (Roche) is the pegylated (i.e. polyethylene glycol modified) form of interferon α-2a. Intron-A® (Schering Corporation) is the recombinant form of Interferon α-2b, and Peg-Intron® (Schering Corporation) is the pegylated form of interferon α-2b.

Other forms of interferon α, as well as interferon (β, γ, τ and ω are currently in clinical development for the treatment of HCV. For example, Infergen® (interferon alphacon-1) by InterMune, Omniferon® (natural interferon) by Viragen, Albuferon® by Human Genome Sciences, Rebif® (interferon (β-1a) by Ares-Serono, Omega Interferon by BioMedicine, Oral Interferon Alpha by Amarillo Biosciences, and interferon γ, interferon τ, and interferon γ-1b by InterMune are in development.

Combination therapy of HCV with ribavirin and interferon-α currently represent the optimal therapy. Combining ribavirin and Peg (infra) results in a sustained virological response (SVR) in 54-56% of patients. The SVR approaches 80% for type 2 and 3 HCV. (Walker, supra) Unfortunately, the combination also produces side effects which pose clinical challenges. Depression, flu-like symptoms and skin reactions are associated with subcutaneous IFN-α and hemolytic anemia is associated with sustained treatment with ribavirin.

A number of potential molecular targets for drug development as anti-HCV therapeutics have now been identified including, but not limited to, the NS2-NS3 autoprotease, the N3 protease, the N3 helicase and the NS5B polymerase. The RNA-dependent RNA polymerase is absolutely essential for replication of the single-stranded, positive sense, RNA genome and this enzyme has elicited significant interest among medicinal chemists.

Nucleoside inhibitors of NS5B polymerase can act either as a non-natural substrate that results in chain termination or as a competitive inhibitor which competes with nucleotide binding to the polymerase. Certain NS5B polymerase nucleoside inhibitors have been disclosed in the following publications, all of which are incorporated by reference in full herein.

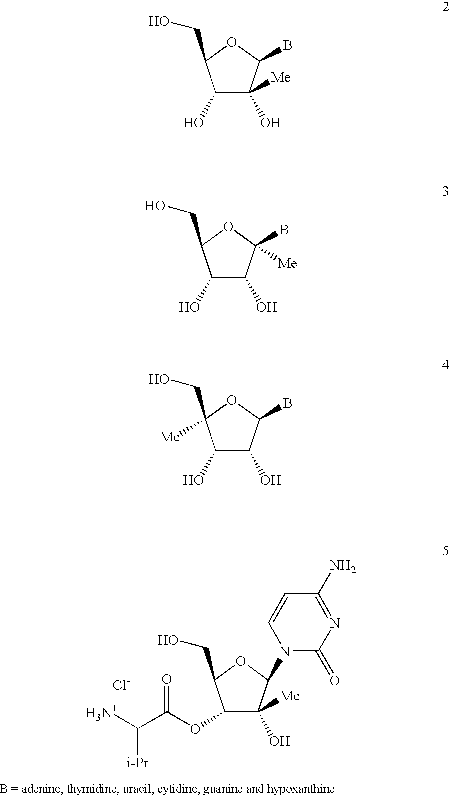

In WO 01 90121 published Nov. 29, 2001, J.-P. Sommadossi and P. Lacolla disclose and exemplify the anti-HCV polymerase activity of 1′-alkyl- and 2′-alkyl nucleosides of formulae 2 and 3. In WO 01/92282, published Dec. 6, 2001, J.-P. Sommadossi and P. Lacolla disclose and exemplify treating Flaviviruses and Pestiviruses with 1′-alkyl- and 2′-alkyl nucleosides of formulae 2 and 3. In WO 03/026675 published Apr. 3, 2003, G. Gosselin discloses 4′-alkyl nucleosides 4 for treating Flaviviruses and Pestiviruses.

In WO2004003000 published Jan. 8, 2004, J.-P. Sommadossi et al. disclose 2′- and 3′ prodrugs of 1′-, 2′-, 3′- and 4′-substituted β-D and β-L nucleosides. In WO 2004/002422 published Jan. 8, 2004, 2′-C-methyl-3′-O-valine ester ribofuransyl cytidine for the treatment of Flaviviridae infections. Idenix has reported clinical trials for a related compound NM283 which is believed to be the valine ester 5 of the cytidine analog 2 (B=cytosine). In WO 2004/002999 published Jan. 8, 2004, J.-P. Sommadossi et al. disclose a series of 2′ or 3′ prodrugs of 1′, 2′, 3′, or 4′ branched nucleosides for the treatment of flavivirus infections including HCV infections.

In WO2004/046331 published Jun. 3, 2004, J.-P. Sommadossi et al. disclose 2′-branched nucleosides and Flaviviridae mutation. In WO03/026589 published Apr. 3, 2003 G. Gosselin et al. disclose methods of treating hepatitis C virus using 4′-modified nucleosides. In WO2005009418 published Feb. 3, 2005, R. Storer et al. disclose purine nucleoside analogues for treatment of diseases caused by Flaviviridae including HCV.

Other patent applications disclose the use of certain nucleoside analogs to treat hepatitis C virus infection. In WO 01/32153 published May 10, 2001, R. Storer discloses nucleosides derivatives for treating viral diseases. In WO 01/60315 published Aug. 23, 2001, H. Ismaili et al., disclose methods of treatment or prevention of Flavivirus infections with nucleoside compounds. In WO 02/18404 published Mar. 7, 2002, R. Devos et al. disclose 4′-substituted nucleotides for treating HCV virus. In WO 01/79246 published Oct. 25, 2001, K. A. Watanabe disclose 2′- or 3′-hydroxymethyl nucleoside compounds for the treatment of viral diseases. In WO 02/32920 published Apr. 25, 2002 and in WO 02/48 165 published Jun. 20, 2002 L. Stuyver et al. disclose nucleoside compounds for the treatment of viral diseases.

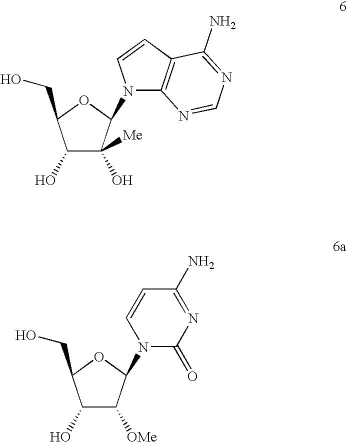

In WO 03/105770 published Dec. 24, 2003, B. Bhat et al. disclose a series of carbocyclic nucleoside derivatives that are useful for the treatment of HCV infections. In WO 2004/007512 published Jan. 22, 2003 B. Bhat et al. disclose nucleoside compounds that inhibit of RNA-dependent RNA viral polymerase. The nucleosides disclosed in this publication are primarily 2′-methyl-2′-hydroxy substituted nucleosides. In WO 2002/057425 published Jul. 25, 2002 S. S. Carroll et al. disclose nucleoside derivatives which inhibitor of RNA-dependent viral polymerase and methods of treating HCV infection. In WO02/057287 published Jul. 25, 2002, S. S. Carroll et al. disclose related 2α-methyl and 2β-methylribose derivatives wherein the base is an optionally substituted 7H-pyrrolo[2,3-d]pyrimidine radical 6. The same application discloses one example of a 3β-methyl nucleoside. S. S. Carroll et al. (J. Biol. Chem. 2003 278(14):11979-11984) disclose inhibition of HCV polymerase by 2′-O-methylcytidine (6a). In WO 2004/009020 published Jan. 29, 2004, D. B. Olsen et al. disclose a series of thionucleoside derivatives as inhibitors of RNA dependent RNA viral polymerase.

PCT Publication No. WO 99/43691 to Emory University, entitled “2′-Fluoronucleosides” discloses the use of certain 2′-fluoronucleosides to treat HCV. U.S. Pat. No. 6,348,587 to Emory University entitled “2′-fluoronucleosides” discloses a family of 2′-fluoronucleosides useful for the treatment of hepatitis B, HCV, HIV and abnormal cellular proliferation. Both configurations of the 2′ fluoro substituent are disclosed.

Eldrup et al. (Oral Session V, Hepatitis C Virus, Flaviviridae; 16^{th} International Conference on Antiviral Research (Apr. 27, 2003, Savannah, Ga.)) described the structure activity relationship of 2′-modified nucleosides for inhibition of HCV.

Bhat et al. (Oral Session V, Hepatitis C Virus, Flaviviridae; 16^{th} International Conference on Antiviral Research (Apr. 27, 2003, Savannah, Ga.); p A75) describe the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

Several classes of non-nucleoside HCV NS5B inhibitors have been described and are incorporated by reference in full herein, including: benzimidazoles, (H. Hashimoto et al. WO 01/47833, H. Hashimoto et al. WO 03/000254, P. L. Beaulieu et al. WO 03/020240 A2; P. L. Beaulieu et al. U.S. Pat. No. 6,448,281 B1; P. L. Beaulieu et al. WO 03/007945 A1); indoles, (P. L. Beaulieu et al. WO 03/0010141 A2); benzothiadiazines, e.g., 7, (D. Dhanak et al. WO 01/85172 A1; D. Dhanak et al. WO 03/037262 A2; K. J. Duffy et al. WO03/099801 A1, D. Chai et al. WO 2004052312, D. Chai et al. WO2004052313, D. Chai et al. WO02/098424, J. K. Pratt et al. WO 2004/041818 A1; J. K. Pratt et al. WO 2004/087577 A1), thiophenes, e.g., 8, (C. K. Chan et al. WO 02/100851);

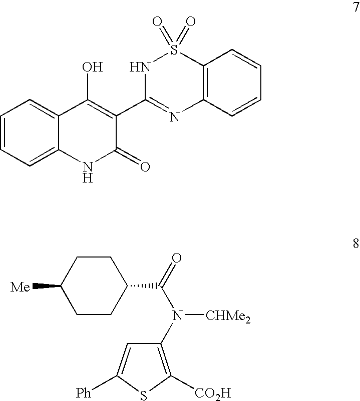

benzothiophenes (D. C. Young and T. R. Bailey WO 00/18231); β-ketopyruvates (S. Attamura et al. U.S. Pat. No. 6,492,423 B1, A. Attamura et al. WO 00/06529); pyrimidines (C. Gardelli et al. WO 02/06246 A1); pyrimidinediones (T. R. Bailey and D. C. Young WO 00/13708); triazines (K.-H. Chung et al. WO 02/079187 A1); rhodanine derivatives (T. R. Bailey and D. C. Young WO 00/10573, J. C. Jean et al. WO 01/77091 A2); 2,4-dioxopyrans (R. A. Love et al. EP 256628 A2); phenylalanine derivatives (M. Wang et al. J. Biol. Chem. 2003 278:2489-2495).

### Nucleoside Prodrugs

Nucleoside derivatives often are potent anti-viral (e.g., HIV, HCV, Herpes simplex, CMV) and anti-cancer chemotherapeutic agents. Unfortunately their practical utility is often limited by two factors. Firstly, poor pharmacokinetic properties frequently limit the absorption of the nucleoside from the gut and the intracellular concentration of the nucleoside derivatives and, secondly, suboptimal physical properties restrict formulation options which could be employed to enhance delivery of the active ingredient. Albert introduced the term prodrug to describe a compound which lacks intrinsic biological activity but which is capable of metabolic transformation to the active drug substance (A. Albert, Selective Toxicity, Chapman and Hall, London, 1951). Produgs have been recently reviewed (P. Ettmayer et al., J. Med. Chem. 2004 47(10):2393-2404; K. Beaumont et al., Curr. Drug Metab. 2003 4:461-485; H. Bundgaard, Design of Prodrugs: Bioreversible derivatives for various functional groups and chemical entities in Design of Prodrugs, H. Bundgaard (ed) Elsevier Science Publishers, Amersterdam 1985; G. M. Pauletti et al. Adv. Drug Deliv. Rev. 1997 27:235-256; R. J. Jones and N. Bischofberger, Antiviral Res. 1995 27; 1-15 and C. R. Wagner et al., Med. Res. Rev. 2000 20:417-45). While the metabolic transformation can catalyzed by specific enzymes, often hydrolases, the active compound can also be regenerated by non-specific chemical processes.

Pharmaceutically acceptable prodrugs refer to a compound that is metabolized, for example hydrolyzed or oxidized, in the host to form the compound of the present invention. The bioconversion should avoid formation fragments with toxicological liabilities. Typical examples of prodrugs include compounds that have biologically labile protecting groups linked to a functional moiety of the active compound. Alkylation, acylation or other lipophilic modification of the hydroxy group(s) on the sugar moiety have been utilized in the design of pronucleotides. These pronucleotides can be hydrolyzed or dealkylated in vivo to generate the active compound.

Factors limiting oral bioavailability frequently are absorption from the gastrointestinal tract and first-pass excretion by the gut wall and the liver. Optimization of transcellular absorption through the GI tract requires a D_{(7.4)} greater than zero. Optimization of the distribution coefficient does not, however, insure success. The prodrug may have to avoid active efflux transporters in the enterocyte. Intracellular metabolism in the enterocyte can result in passive transport or active transport of the metabolite by efflux pumps back into the gut lumen. The prodrug must also resist undesired biotransformations in the blood before reaching the target cells or receptors.

While putative prodrugs sometimes can rationally designed based on the chemical functionality present in the molecule, chemical modification of an active compound produces an entirely new molecular entity which can exhibit undesirable physical, chemical and biological properties absent in the parent compound. Regulatory requirements for identification of metabolites may pose challenges if multiple pathways lead to a plurality of metabolites. Thus, the identification of prodrugs remains an uncertain and challenging exercise. Moreover, evaluating pharmacokinetic properties of potential prodrugs is a challenging and costly endeavor. Pharmacokinetic results from animal models may be difficult to extrapolate to humans.

### SUMMARY OF THE INVENTION

The present invention is based on the discovery that in patients infected with Genotype 1 of the Hepatitis C virus (HCV-1) or Genotype 4 HCV (HCV-4), a beneficial response to a treatment that includes interferon alpha, ribavirin and a HCV polymerase inhibitor could be predicted if the patient's HCV RNA level becomes undetectable in as short as two weeks post treatment. In one embodiment, the invention provides for a method for predicting response of a human subject infected with HCV-1 to a treatment with interferon, ribavirin and a HCV NS5B polymerase inhibitor comprising providing a sample from the subject at around Week 2 of treatment and determining the level of HCV-1 or HCV-4 RNA in the sample wherein an undetectable level of HCV-1 or HCV-4 RNA in the sample indicates a likelihood of sustained virological response achieved by the subject to the treatment. In another embodiment, the invention provides for a method for selecting a duration of treatment with interferon, ribavirin and a HCV NS5B polymerase inhibitor for achievement of sustained virological response in a human subject infected with HCV-1 or HCV-4 comprising providing a sample from the subject at around Week 2 of the treatment and determining the level of HCV-1 or HCV-4 RNA in the sample wherein an undetectable level of HCV-1 or HCV-4 RNA in the sample indicates a duration of between 8 weeks and 12 weeks of treatment for achievement of sustained virological response in the subject.

### BRIEF DESCRIPTION OF THE DRAWINGS

FIG. 1 shows the Study Design of the Phase II Clinical Trial for RO4588161

FIG. 2 shows the individual HCV RNA response pattern over time in the patients receiving the triple treatment of 1500 mg RO4588161, Pegasys and Ribavirin.

FIG. 3 is a graphical representation of the HCV RNA levels of patients receiving 1500 mg RO5024048 with Pegasys and Ribavirin whose HCV RNA became undetectable before or at 14 days of treatment. HCV RNA level is shown on the y-axis as log₁₀ IU/ml.

FIG. 4 is a graphical representation of the HCV RNA levels of patients receiving 1500 mg RO5024048 with Pegasys and Ribavirin whose HCV RNA became undetectable after 14 days but before or at 17 days of treatment. HCV RNA level is shown on the y-axis as log₁₀ IU/ml.

FIG. 5 is a graphical representation of the HCV RNA levels of patients receiving 1500 mg RO5024048 with Pegasys and Ribavirin whose HCV RNA were undetectable after 17 days but before or at 28 days of treatment. HCV RNA level is shown on the y-axis as log₁₀ IU/ml.

### DETAILED DESCRIPTION OF THE INVENTION

### Definitions

The term “response” to treatment with interferon is a desirable response to the administration of an agent. The terms “Sustained Virologic Response” and “Complete Response” to treatment with interferon are herein used interchangeably and refer to the absence of detectable HCV RNA (<15 IU/mL) in the sample of an infected subject by RT-PCR both at the end of treatment and twenty-four weeks after the end of treatment. The terms “Virologic Non-Response” and “No Response” to treatment with interferon are herein used interchangeably and refer to the presence of detectable HCV RNA (>=15 IU/mL) in the sample of an infected subject by RT-PCR throughout treatment and at the end of treatment.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

The terms “interferon” and “interferon-alpha” are used herein interchangeably and refer to the family of highly homologous species-specific proteins that inhibit viral replication and cellular proliferation and modulate immune response. Typical suitable interferons include, but are not limited to, recombinant interferon alpha-2b such as Intron® A interferon available from Schering Corporation, Kenilworth, N.J., recombinant interferon alpha-2a such as Roferon®-A interferon available from Hoffmann-La Roche, Nutley, N.J., recombinant interferon alpha-2C such as Berofor® alpha 2 interferon available from Boehringer Ingelheim Pharmaceutical, Inc., Ridgefield, Conn., interferon alpha-n1, a purified blend of natural alpha interferons such as Sumiferon® available from Sumitomo, Japan or as Wellferon® interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred. Interferons can include pegylated interferons as defined below.

The terms “pegylated interferon”, “pegylated interferon alpha” and “peginterferon” are used herein interchangeably and means polyethylene glycol modified conjugates of interferon alpha, preferably interferon alpha-2a and alpha-2b. Typical suitable pegylated interferon alpha include, but are not limited to, Pegasys® and Peg-Intron®.

The term “ribavirin” refers to the compound, 1-((2R,3R,4S,5R)-3,4-Dihydroxy-5-hydroxymethyl-tetrahydro-furan-2-yl)-1H-[1,2,4]triazole-3-carboxylic acid amide which is a synthetic, non-interferon-inducing, broad spectrum antiviral nucleoside analog and available under the names, Virazole® and Copegus®.

The term “RO4588161” as used herein refers to the compound, Isobutyric acid (2R,3S,4R,5R)-5-(4-amino-2-oxo-2H-pyrimidin-1-yl)-2-azido-3,4-bis-isobutyryloxy-tetrahydro-furan-2-ylmethyl ester, including pharmaceutically acceptable acid addition salts, and is used interchangeably with the term “R1626” as disclosed in P. J. Pockros et al., Hepatology, 2008, 48: 385-397, which is incorporated by reference in full herein.

The term “RO5024048” as used herein refers to the compound, Isobutyric acid (2R,3R,4R,5R)-5-(4-amino-2-oxo-2H-pyrimidin-1-yl)-4-fluoro-3-isobutyryloxy-4-methyl-tetrahydro-furan-2-ylmethyl ester, including pharmaceutically acceptable acid addition salts, and is used interchangeably with the term “R7128” as disclosed in S. Ali et al., Antimicrob Agents Chemother., 2008 52(12):4356-4369, which is incorporated by reference in full herein. The term “NM283” and “valopicitabine” are used herein interchangeably and refers to the compound, 3′-O-(L-valinyl)-2′-C-methyl-β-D-cytidine, including pharmaceutically acceptable acid addition salts, as disclosed in C. Pierra et al., J. Med. Chem., 2006, 49(22):6614-6620, which is incorporated by reference in full herein.

The term “MK-0608” as used herein refers to the compound, 2′-C-methyl-7-deaza-adenosine, including pharmaceutically acceptable acid addition salts, as disclosed in D. B. Olsen et al., Antimicrob Agents Chemother., 2004, 48:3944-3953, which is incorporated by reference in full herein.

The term “around Week 2” refers to a time period of two weeks or fourteen days, plus or minus 1 to 2 days.

The current recommended first line treatment for patients with chronic hepatitis C is pegylated interferon alpha in combination with ribavirin for 48 weeks in patients carrying genotype 1 or 4 virus and for 24 weeks in patients carrying genotype 2 or 3 virus. Combined treatment with ribavirin was found to be more effective than interferon alpha monotherapy in patients who relapsed after one or more courses of interferon alpha therapy, as well as in previously untreated patients. However, ribavirin exhibits significant side effects including teratogenicity and carcinogenicity. Furthermore, ribavirin causes hemolytic anemia requiring dose reduction or discontinuation of ribavirin therapy in approximately 10 to 20% of patients, which may be related to the accumulation of ribavirin triphosphate in erythrocytes. Therefore, to reduce treatment cost and the incidence of adverse events, it is desirable to tailor the treatment to a shorter duration while not compromising efficacy.

Numerous studies have shown that rapid virological response (RVR) at 4 weeks has been a fairly reliable predictor of a sustained virological response (SVR) for treatment using peginterferon/ribavarin. Some studies have shown that among HCV-1 patients that achieve RVR, the SVR rates were comparable between 24-week and 48-week peginterferon/ribovarin treatment (D. M. Jensen et al., Hepatology, 2006, 43:954-960; S. Zeuzen et al., J. Hepatol. 2006, 44:97-103; A. Mangia et al., Hepatology, 2008, 47: 43-50), while others demonstrate that even if RVR is attained, 24 weeks of peginterferon/ribavirin is inferior to 48 weeks of treatment in HCV-1 patients (M.-L. Yu et al., Hepatology, 2008, 47:1884-1893.

### Examples

### Phase II Clinical Trial Involving RO4588161

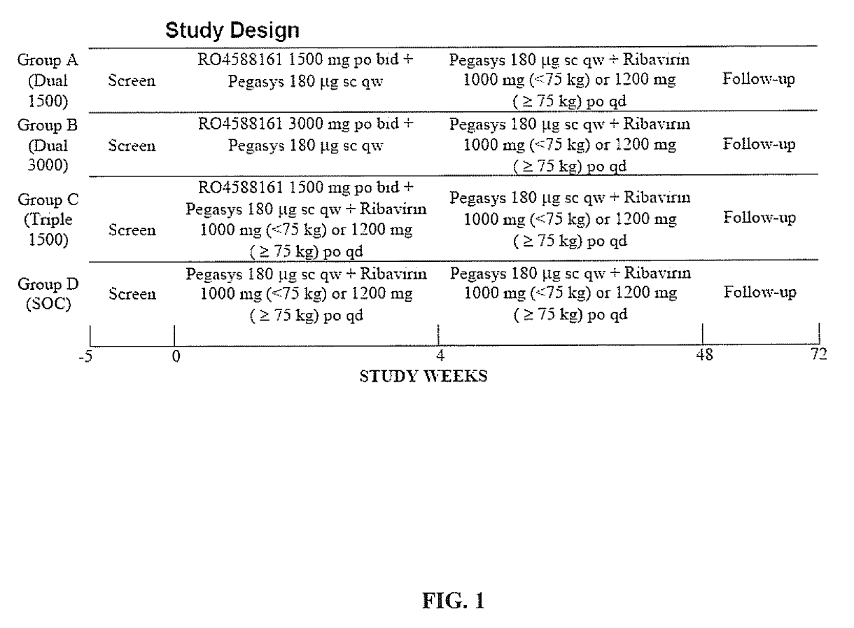

This was a phase 2A, multi-center, randomized, double-blinded (RO4588161 and ribavirin were double-blinded and Pegasys was open labeled), active-controlled, with a parallel-group study which is ongoing. A screening period (time from the first screening assessment to the first administration of test drug) of 35 days preceded the treatment portion of the trial (FIG. 1). The HCV genotype and HCV RNA titer of each patient was confirmed during the screening period and only treatment-naïve patients with HCV genotype-1 and HCV RNA titer 50,000 IU/mL were eligible for enrollment.

One hundred and seven male and female patients between 18 and 66 years of age were enrolled into the study. Patients were randomized into four treatment groups:
Group A/Dual 1500 [RO4588161 1500 mg oral, twice daily+Pegasys 180 μg subcutaneous, once weeky] for 4 weeks-21 patients,
Group B/Dual 3000 [RO4588161 3000 mg oral, twice daily+Pegasys 180 μg subcutaneous, once weekly] for 4 weeks-34 patients,
Group C/Triple 1500 [RO4588161 1500 mg oral, twice daily+Pegasys 180 μg subcutaneous, once weekly+ribavirin 1000 mg (<75 kg) or 1200 mg (75 kg) oral daily] for 4 weeks-31 patients or
Group D/standard of care (SOC) [Pegasys 180 μg subcutaneous, once weekly+ribavirin 1000 mg (<75 kg) or 1200 mg (≧75 kg) oral daily] for 4 weeks-21 patients

From a total of 107 patients, data from 104 patients was evaluable for analysis since 3 patients though randomized did not receive a single dose of study medication. Among the 104 patients there were a total of 43, 4, and 5 patients who prematurely withdrew for safety reasons from RO4588161, Pegasys, and ribavirin treatment, respectively.

Patients meeting all eligibility criteria were randomized to receive RO4588161 in combination with Pegasys with or without ribavirin for 4 weeks or to SOC.

All patients who received at least one dose of study medication would continue to receive open label Pegasys 180 μg sc qw and ribavirin 1000 mg (<75 kg) or 1200 mg (≧75 kg) po qd to complete a total treatment period of 48 weeks.

Randomization was stratified by the PK subcohort (sparse PK versus intensive PK) in a 2:3:3:2 ratio into the following treatment groups (Group A/Dual 1500˜20, Group B/Dual 3000˜30, Group C/Triple 1500˜30, Group D/SOC˜20).

All patients were to have a safety follow up visit at week 8, 4 weeks after the last dose of the experimental drug combination. Patients were to have this 4 week safety follow up visit during their treatment with the standard of care therapy. Patients who have completed a full 48-week course of therapy were followed for 24 weeks post treatment completion.

Pharmacodynamic analysis included the assessment of serum viral load, and viral response at individual clinical visits and an assessment of antiviral resistance development with RO4588161 given in combination with Pegasys with or without ribavirin in treatment naïve patients with chronic HCV genotype 1 virus infection. Viral response was defined as the percentage of patients with undetectable HCV RNA as measured by the Roche COBAS TaqMan HCV Test (<15 IU/mL). Pharmacodynamic data were presented by listings, summary statistics (including means, medians, standard errors, confidence intervals for means, ranges, coefficients of variation, proportions of patients with response and confidence intervals for proportions) and plots of means over time.

### Results

Dose- and time-dependent decreases in plasma viral load were observed following treatment with RO4588161, Pegasys and ribavirin. Declines in HCV RNA were observed as early as the first assessment (72 hours) following the first dose. All RO4588161 containing groups had ≧3.6 log₁₀ decrease in the mean HCV RNA (IU/mL) from baseline at week 4, all larger than 2.4 log₁₀ with SOC. Dual 1500 and Dual 3000 revealed dose dependent decreases with a difference in mean change in viral concentrations of minus 0.9 log₁₀ IU/mL (−3.6 vs.-4.5). When comparing Dual 1500 and Triple 1500 (same dose of RO4588161 and Pegasys, but with ribavirin), the difference was even greater at minus 1.6 log₁₀ IU/mL (−5.2 vs.-3.6). In addition, when comparing SOC and Triple 1500 (same dose of Pegasys and ribavirin, but with RO4588161), the difference was the most pronounced at minus 2.8 log₁₀ IU/mL (−5.2 vs. −2.4). In addition, the 95% confidence intervals between Triple 1500 and Dual 1500, and between Triple 1500 and SOC were all non-overlapping, indicating a superior antiviral effect of Triple 1500 over Dual 1500 and SOC.

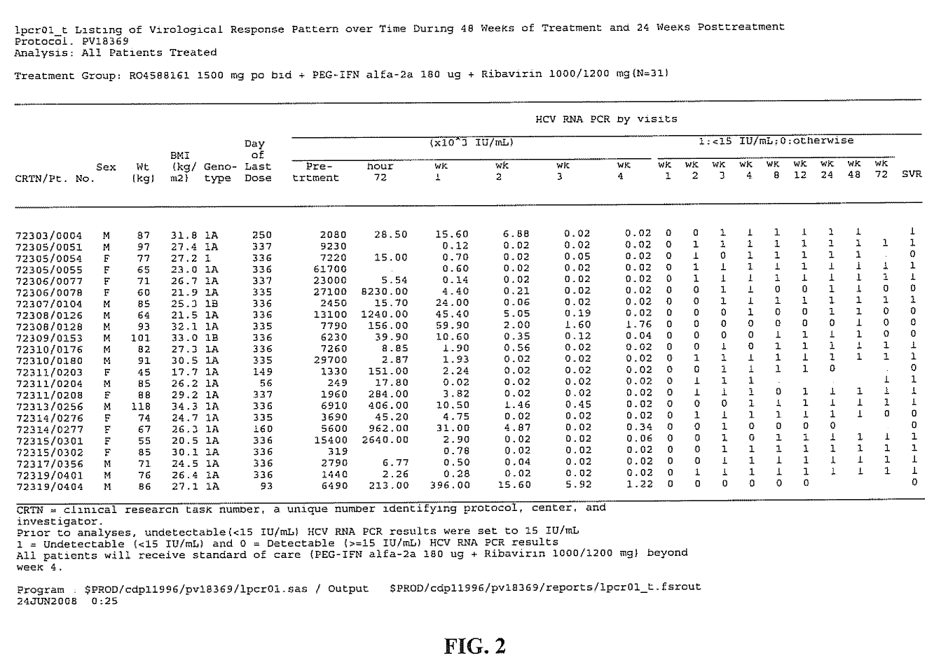

From the individual patient perspective, the change in the pattern of antiviral effect was primarily driven by a number of patients in Triple 1500 who were HCV RNA undetectable at or near the end of the 4 week treatment and subsequently became detectable between the week 4 and week 8. FIG. 2 offers individual HCV RNA response (<15 IU/mL) patterns over time for the Triple 1500 group (N=31). Thirteen out of the 31 patients had undetectable HCV RNA at two weeks of treatment. Eleven of them were able to achieve sustained virological response (SVR) at 24 weeks post treatment completion. In comparison, 23 out of the 31 patients achieved RVR4 (Rapid Virological Response at week 4) or had undetectable HCV RNA at four weeks of treatment. Only 16 out of these 23 patients achieved SVR. Finally, out of the twenty patients treated with only Pegasys and ribovarin, only ten achieved sustained virological response and none of them had HCV RNA that was undetectable at Week 2 of treatment.

### Phase II Clinical Trial Involving RO5024048

Up to 75 treatment-naïve patients with HCV genotype 1 infection were enrolled in 3 cohorts using three dose levels of RO5024048 (0 mg, 500 mg, or 1500 mg oral, twice daily) in combination with standard doses of Pegasys (180 μg subcutaneous, once weekly) and ribovarin [1000 mg (<75 kg) or 1200 mg 75 kg) oral daily], and up to 25 non-responder patients with HCV genotype 2 or 3 infection (e.g., subjects who did not achieve sustained virological response after prior pegylated interferon therapy, and did not discontinue for tolerability or toxicity reasons) may be enrolled to study a dose level of RO5024048 in combination with Pegasys and ribovarin. Twenty-five (25) patients per Cohort of RO5024048 were enrolled. Twenty (20) patients per cohort received RO5024048 in combination with standard of care (SOC) and five (5) patients per cohort were randomized to receive SOC with a RO5024048 placebo. Patients were screened up to 56 days prior to enrollment. On Day −1, patients visited the clinic for pre-dose assessments. On Day 1, patients received an oral dose of study drug RO5024048 or placebo and Pegasys/ribovarin in the morning with a light meal and at least 240 mL of water.

A majority of dosing occurred on an outpatient basis. Periodically, patients returned to the clinic for study assessments. Early morning on Days 7, 14, 21, and 28, patients were asked to report to the clinic after an overnight fast. Patients received their RO5024048/placebo in combination with Pegasys and Ribovarin with a light meal and 240 mL of water in the morning in the clinic. Patients were followed through Day 56 (28 days after dosing is completed).

After completing four weeks of the combination-dosing regimen followed by at least 4 weeks of SOC dosing (RO5024048 washout), all patients were eligible to receive up to 40 weeks, depending upon genotype, of open label standard of care (SOC) dosing with Pegasys plus ribavirin. Genotype 2 or 3 subjects enrolling in Cohort 4 will receive a total of 24 weeks of Pegasys plus ribovarin. An overview of the study design is outlined in Table 1.

TABLE 1

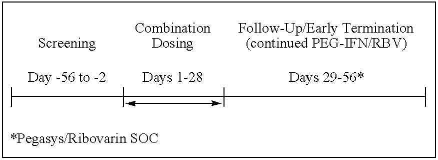

### Results

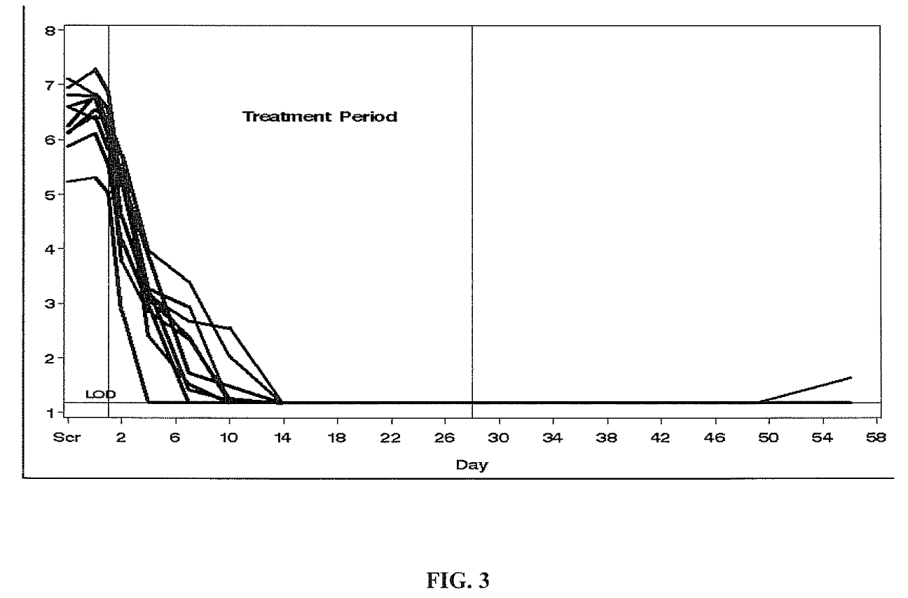

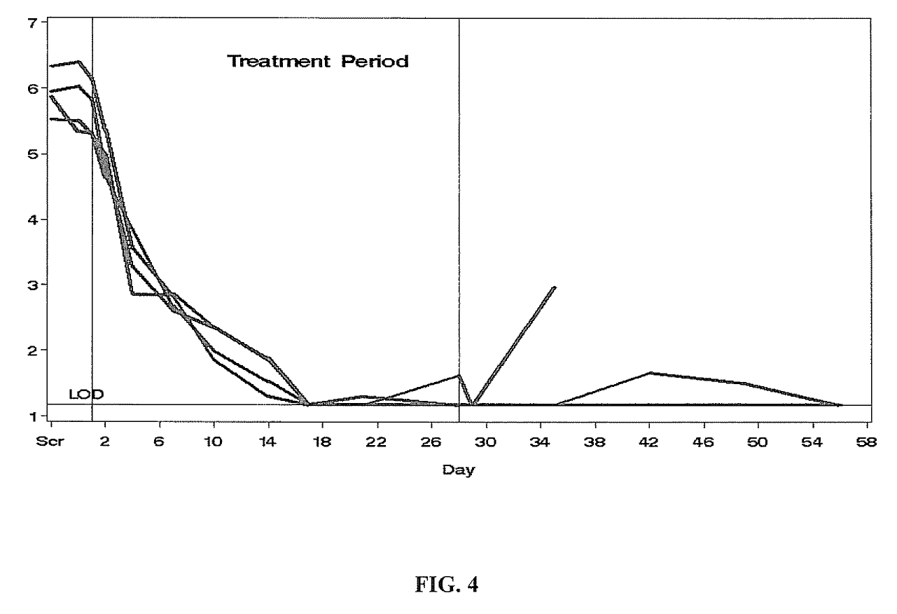

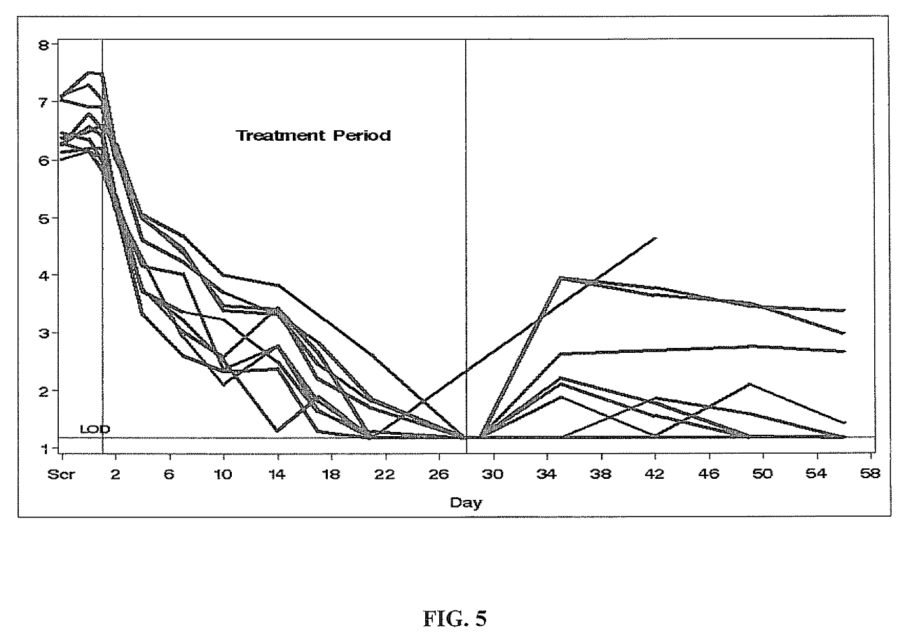

The most notable decreases in in plasma viral load were detected in patients receiving a four-week treatment dose of 1500 mg RO5024048 twice daily together with SOC dosing of Pegasys and ribovarin. FIG. 3 shows that patients who had undetectable HCV RNA levels at 14 days of treatment (Week 2) were all HCV RNA negative when measured on day 56 (Week 8). In contrast, patients who did not exhibit undetectable HCV RNA until day 17 of treatment (FIG. 4) or day 28 of treatment (FIG. 5) were mixed with respect to whether or not HCV RNA could be detected at Week 8.

1. A method for predicting response of a human subject infected with Hepatitis C Virus Genotype 1 (HCV-1) or Hepatitis C Virus Genotype 4 (HCV-4) to a treatment with interferon, ribavirin and a HCV NS5B polymerase inhibitor comprising:
providing a sample from said subject at around Week 2 of said treatment and
determining the level of HCV-1 or HCV-4 RNA in said sample wherein an undetectable level of HCV-1 or HCV-4 RNA in said sample indicates a likelihood of sustained virological response achieved by said subject to said treatment.

2. The method of claim 1 wherein said treatment comprises a dose of 180 micrograms weekly of interferon, 1000 milligrams or 1200 milligrams daily of ribavirin and 1500 milligrams daily of the HCV NS5B polymerase inhibitor.

3. The method of claim 2 wherein said interferon is selected from the group consisting of Pegasys® (peginterferon alpha-2a), Peg-Intron® (peginterferon alpha-2b), Roferon-A® (interferon alpha-2a) and Intron A® (interferon alpha-2b) and said HCV NS5B polymerase inhibitor is selected from the group consisting of RO4588161, RO 5024048, NM283, and MK-0608.

4. The method of claim 3 wherein said HCV NS5B polymersase inhibitor is selected from RO4588161 or RO5024048.

5. A method for selecting a duration of treatment with interferon, ribavirin and a HCV NS5B polymerase inhibitor for achievement of sustained virological response in a human subject infected with HCV-1 or HCV-4 comprising:
providing a sample from said subject at around Week 2 of said treatment and
determining the level of HCV-1 or HCV-4 RNA in said sample wherein an undetectable level of HCV-1 or HCV-4 RNA in said sample indicates a duration of between 8 weeks and 12 weeks of said treatment for achievement of sustained virological response in said subject.

6. The method of claim 5 wherein said treatment comprises a dose of 180 micrograms weekly of interferon, 1000 milligrams or 1200 milligrams daily of ribavirin and 1500 milligrams daily of the HCV NS5B polymerase inhibitor.

7. The method of claim 6 wherein said interferon is selected from the group consisting of Pegasys® (peginterferon alpha-2a), Peg-Intron® (peginterferon alpha-2b), Roferon-A® (interferon alpha-2a) and Intron A® (interferon alpha-2b) and said HCV NS5B polymerase inhibitor is selected from the group consisting of RO4588161, RO 5024048, NM283, and MK-0608.

8. The method of claim 3 wherein said HCV NS5B polymersase inhibitor is selected from RO4588161 or RO5024048.
